(19) United States
(12) Patent Application Publication
(10) Pub. No.: US 2017/0367634 A1
(43) Pub. Date: Dec. 28, 2017

(54) METHOD AND SYSTEM FOR EMOTION MAPPING

(71) Applicants: Rita H. Wouhaybi, Portland, OR (US); Lama Nachman, Santa Clara, CA (US); Sangita Sharma, Portland, OR (US); Giuseppe Raffa, Portland, OR (US)

(72) Inventors: Rita H. Wouhaybi, Portland, OR (US); Lama Nachman, Santa Clara, CA (US); Sangita Sharma, Portland, OR (US); Giuseppe Raffa, Portland, OR (US)

(21) Appl. No.: 15/191,997

(22) Filed: Jun. 24, 2016

(51) Int. Cl. A61B 5/16; A61B 5/00; A61B 5/0205; H04L 29/08; A61B 5/024

(52) U.S. Cl. CPC A61B 5/165; A61B 5/02055; H04L 67/306; A61B 5/7278; H04L 67/12; H04L 67/18; A61B 5/0059; A61B 5/02438; A61B 2560/0252; A61B 2562/0204; A61B 2562/029; A61B 2560/0257

(57) ABSTRACT

A system for mapping user emotions includes a plurality of sensors for measuring user emotion indications and environmental data. A controller is coupled to the plurality of sensors. The controller reads the plurality of sensors to obtain at least one user emotion indication and environmental data coincident with the at least one user emotion indication. The controller further associates the environmental data with the user emotion indication.

### TECHNICAL FIELD

Embodiments described herein pertain in general to emotion mapping and in particular to capturing user emotion indications and tagging them with geographical location and activity associated with the emotion indications.

### BACKGROUND

Emotions are important for the well-being of all individuals. We are not only affected by our own emotions but by the emotions of those around us, even those we do not interact directly with.

Research has been conducted to understand the capacity of individuals to recognize their own, and other people's emotions, to discriminate between different feelings and label them appropriately, and to use emotional information to guide thinking and behavior. This is typically referred to as Emotional Intelligence (EI) or the Emotional Quotient (EQ). The findings have been valuable but have only scratched the surface of an important topic. Eventually the research will come to a scalability barrier where findings are based only on a small subset of the population.

### BRIEF DESCRIPTION OF THE DRAWINGS

FIG. 1 illustrates a block diagram of an emotion mapping system for mapping human emotion, according to various embodiments.

FIG. 2 illustrates a block diagram of a computer operating as a controller device or computer server of the emotion mapping system, according to various embodiments.

FIG. 3 illustrates a flowchart of a method for emotion mapping, according to various embodiments.

FIG. 4 illustrates a flow diagram of a method for emotion mapping, according to various embodiments.

### DETAILED DESCRIPTION

As wearable electronic devices (e.g., smart watches, glasses) and mobile devices (e.g., smartphones) become more prevalent in society, the sensors of these devices may be used to detect the user's emotional state and sentiment. For example, texts generated by a user, audio feedback and user conversations, user heart rate, user galvanic skin reaction, as well as other user vital signs may be monitored by various sensors as emotion indications. The emotion indications may then be used to determine a user's positive or negative reaction to some stimulus (e.g., event).

The emotion indications may be tagged (i.e., associated) with environmental data collected from sensors associated with the user and coincident with the emotion that generated the emotion indication. The emotion indications and their associated environmental data may then be aggregated and displayed in various ways. For example, a heatmap or temporal map may be used to display the emotional signature of a location over time.

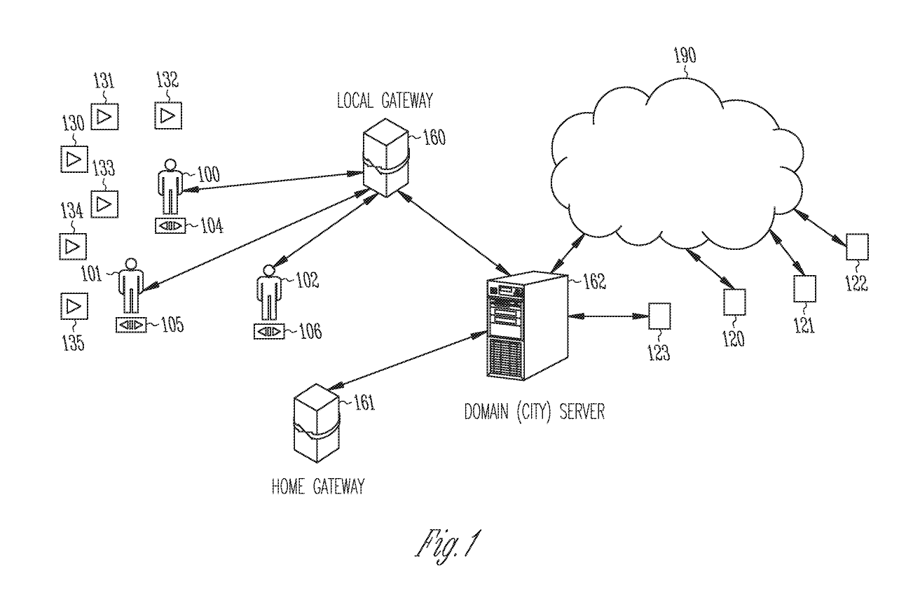

FIG. 1 illustrates a block diagram of an emotion mapping system for mapping human emotion, according to an embodiment. This system is for purposes of illustration only as other systems may be used to collect emotion indications and environmental data associated with those emotion indications.

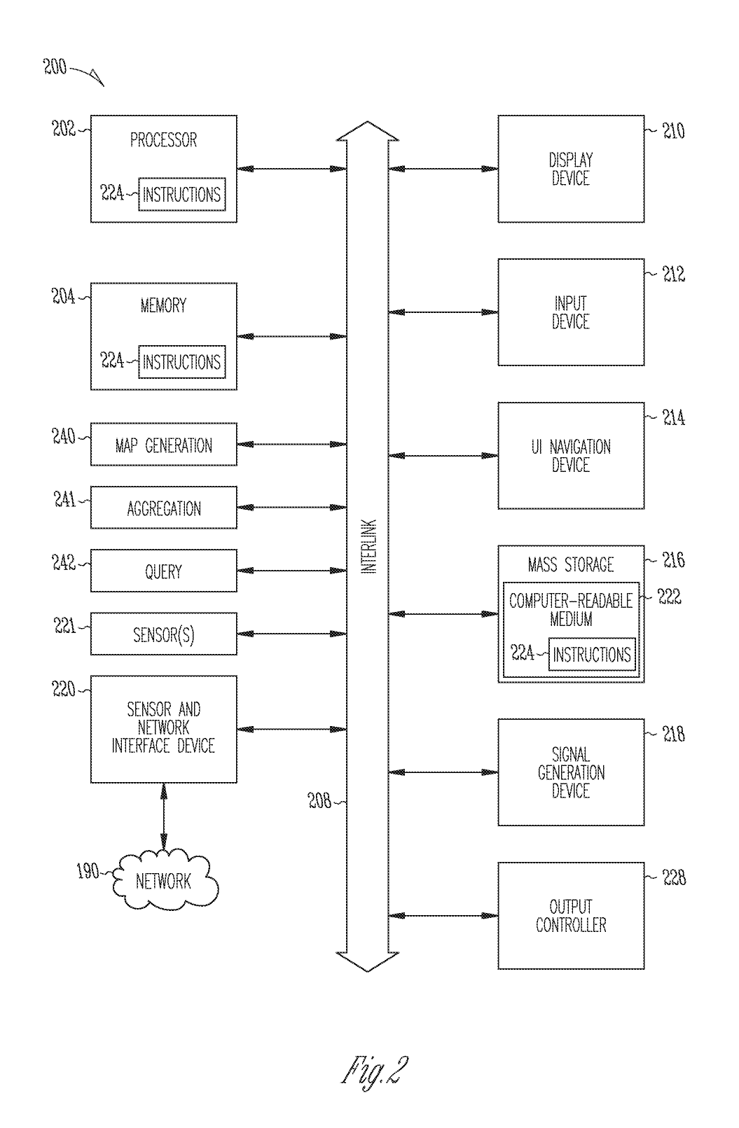

The system is being used by a plurality of users 100-102. Each user 100-102 may be using or be associated with a controller device 104-106 (e.g., electronic device, computer) that includes or is coupled to one or more sensors. For example, the controller devices 104-106 may include smartphones, smartwatches, electronic glasses, or other mobile electronic devices. One embodiment of a controller device 104-106 is illustrated in FIG. 2.

The controller devices 104-106 comprise or are coupled to a plurality of sensors. For example, the sensors may include vital sign monitors (e.g., skin/body thermometer, heart rate monitors), galvanic skin monitors, image sensors, microphones, tactile, or other types of emotion sensors to monitor human emotion indications and/or reactions to an event.

The emotion indications may be defined as any user action, inaction, biological reading, audio response, typed message, or any other indication of the user's emotional state at a particular moment in time. For example, a tactile sensor may monitor what the user is typing in response to an event, a microphone may monitor what the user says in response to the event, a heart rate sensor may monitor the user's heartbeat in response to the event, and/or an imaging device may monitor the user's facial expression in response to an event. The data from each of these sensors may be used as an indication of the user's emotional state such as a positive emotional state for positive indication or a negative emotional state for negative indications. Additionally, the intensity of the emotional state may be determined (e.g., a relatively higher heart rate, a relatively louder audio response) and stored. The controller device 104-106 may then store indications of these responses in the controller device memory or elsewhere with environmental data coincident with that event in order to show the user's response to that particular event.

As an example of emotion indication monitoring, a smartwatch may contain a heart rate monitor to measure the user's heart rate. Heart rate variability may be used to monitor the user's emotional status. A high heart rate may be an indication of the user being excited, nervous, angry, happy, or anxious about an event. While the heart rate may increase for any of these emotions, the amount of time that the heart rate is elevated and the length of time it takes for the heart rate to recover to normal for that user may differentiate between a positive emotion and a negative emotion experienced by the user. The other emotion sensors may be used in a substantially similar way.

Environmental sensors 130-135 may be positioned around the users 100-102 and used to record environmental data coincident, in time and/or space, with the emotion indications recorded by the emotional sensors. The environmental sensors 130-135 may include any physical environment sensors not used to determine the emotional condition of the user. For example, the environmental sensors 130-135 may include air quality, temperature, humidity, image, sound, light, or barometric sensors. In another embodiment, the controller devices 104-106 may also include or be coupled to one or more of the environmental sensors.

As used herein, the environmental data coincidental with the emotion indication may be defined as geographical location (e.g., determined by a GPS sensor), temperature, humidity, air quality, time, news read by the user, messages (e.g., texts, emails) generated or read by the user, online content that the user is exposed to, ambient sounds around the user, or any other measureable event that may affect the user.

Various computer servers 160-162 may be used to couple the controller devices 104-106 to a network 190 (e.g., Internet, local area network (LAN), wide area network (WAN) over a wired or wireless channel. For example, the user controller devices 104-106 may be coupled to and communicate with the computer servers 160-162 over one set of wireless channels and the computer servers 160-162 may be coupled to the network 190 over a second set of wireless channels. Thus, the computer servers 160-162 may be used to aggregate the emotion indications with their respective associated environmental data and store it either locally on the computer servers 160-162 or share it with other locations over the network 190 based on user privacy settings associated with each user. The computer servers 160-162 may also be functionally referred to herein as controllers or gateways.

Each users' controller device 104-106 may register itself with a local gateway server 160 and request copies of the environmental data associated with certain recorded emotion indications. The local gateway server 160 may perform an authentication process, for security reasons, in order to verify that the requested data belongs to the user requesting it. The controller device 104-106 may include privacy settings, set by the user, to prevent disclosure of certain data to the local gateway server 160 such as which applications are being executed by the users' controller device 104-106 or how the requested data is going to be used by the controller device 104-106.

When the user arrives at home or to a trusted environment, the data exchanged with a home computer server 161 (i.e., home gateway) may appear different where the sensing is more collaborative and the data sharing enabled. The home gateway 161 may be located in the same location where the user's data is captured, saved, and analyzed to create the local views that may be queried by applications. The home gateway 161 may serve controller devices 104-106 associated with one user 102 or a household of users 100-102 and act as a data storage device for users. The home gateway 161 may be used to tunnel data to other servers when the user is in other locations. Similarly, the local gateway 160 may be a computer server with a location context that can collect data related to a location, thus creating a sentiment signature of that location. This may be aggregated among multiple computer servers 160-162 based on location or other contexts. For example, a domain server 162 may aggregate data based on one or multiple contexts.

When one or more of the computer servers 160-162 collects data from the user controller devices 104-106, one or more of a plurality of executing applications 120-123 may also be coupled to the network 190 or to one or more of the computer servers 160-162 to access the emotion indications with their respective associated environmental data. These applications 120-121 may be able to filter and aggregate the data in various ways in order to display the emotion indications according to selected environmental data. Users 100-102 and gateway owners may be able to set privacy policies on how the data is distributed with various entities. They may also be able to set sampling rate of the data, specific contexts (e.g., locations, people), aggregation with other data, or removal of particular incidents. Potentially, users and gateway owners may be able to provide fraudulent data as well.

FIG. 2 illustrates a block diagram of a computer 200 operating as a controller device 104-106 or a computer server 160-162 of the emotion mapping system according to an embodiment. The computer 200 may operate as a standalone device or may be coupled (e.g., networked) to other computers. The computer may be referred to as an emotion mapping module to execute any methods disclosed herein.

In a networked deployment, the computer 200 may operate in the capacity of a server computer, gateway, and/or a client computer in server-client network environments. In an example, the computer 200 may act as a peer computer in peer-to-peer (P2P) (or other distributed) network environment.

The computer 200 may be a personal computer (PC), a tablet PC, a set-top box (STB), a Personal Digital Assistant (PDA), a mobile telephone, a smartphone, a web appliance, a network router, switch or bridge, or any computer capable of executing instructions (sequential or otherwise) that specify actions to be taken by that computer. Further, while a single computer is illustrated, the term “computer” shall also be taken to include any collection of computers that individually or jointly execute a set (or multiple sets) of instructions to perform any one or more of the methodologies discussed herein, such as obtaining indications of user emotions and environmental data or aggregating the user emotion indications based on environmental data associated with each user emotion indication.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) a specified manner as a module. In an example, at least a part of one or more computer systems (e.g., a standalone, client or server computer system) or one or more hardware processors 202 may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform at least part of any operation described herein. Considering examples in which modules are temporarily configured, a module need not be instantiated at any one moment in time. For example, where the modules comprise a general-purpose hardware processor 202 configured using software; the general-purpose hardware processor may be configured as respective different modules at different times. Software may accordingly configure a hardware processor, for example, to constitute a particular module at one instance of time and to constitute a different module at a different instance of time. The term “application,” or variants thereof, is used expansively herein to include routines, program modules, programs, components, and the like, and may be implemented on various system configurations, including single-processor or multiprocessor systems, microprocessor-based electronics, single-core or multi-core systems, combinations thereof, and the like. Thus, the term application may be used to refer to an embodiment of software or to hardware arranged to perform at least part of any operation described herein.

The computer (e.g., controller device, computer, server, electronic device) 200 may include a hardware processor 202 (e.g., a central processing unit (CPU), a graphics processing unit (GPU), a hardware processor core, or any combination thereof), memory 204, at least some of which may communicate with others via an interlink (e.g., bus) 208. The computer 200 may further include a display unit 210 (e.g., means for displaying), an alphanumeric input device 212 (e.g., a keyboard), and a user interface (UI) navigation device 214 (e.g., a mouse). In an example, the display unit 210, input device 212 and UI navigation device 214 may be a touch screen display. The computer 200 may additionally include a storage device (e.g., drive unit) 216, a signal generation device 218 (e.g., a speaker), a network interface device 220, and one or more sensors 221, such as emotional sensors, environmental sensors, a global positioning system (GPS) sensor, compass, accelerometer, or other sensors or sensor interfaces. The computer 200 may include an output controller 228, such as a serial (e.g., universal serial bus (USB), parallel, or other wired or wireless (e.g., infrared (IR)) connection to communicate or control one or more peripheral devices (e.g., a printer, card reader, etc.

The storage device 216 may include at least one transitory or non-transitory computer-readable medium 222 on which is stored one or more sets of data structures or instructions 224 (e.g., software) embodying or utilized by any one or more of the techniques or functions described herein. The instructions 224 may also reside, at least partially, in additional computer-readable memories such as memory 204 or within the hardware processor 202 during execution thereof by the machine 200. In an example, one or any combination of the hardware processor 202, the memory 204 or the mass storage device 216 may constitute transitory or non-transitory computer-readable media.

While the computer-readable medium 222 is illustrated as a single medium, the term “machine readable medium” may include a single medium or multiple media (e.g., a centralized or distributed database, and/or associated caches and servers) that are configured to store the one or more instructions 224.

The term “computer-readable medium” may include any medium that is capable of storing, encoding, or carrying instructions for execution by the computer 200 and that cause the computer 200 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed computer readable medium comprises a computer readable medium with a plurality of particles having resting mass. Specific examples of massed computer-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 224 may further be transmitted or received over a communications network 190 using a transmission medium via the network interface device 220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks ((e.g., channel access methods including Code Division Multiple Access (CDMA), Time-division multiple access (TDMA), Frequency-division multiple access (FDMA), and Orthogonal Frequency Division Multiple Access (OFDMA) and cellular networks such as Global System for Mobile Communications (GSM), Universal Mobile Telecommunications System (UMTS), CDMA 2000 1x* standards and Long Term Evolution (LTE)), Plain Old Telephone (POTS) networks, and wireless data networks (e.g., Institute of Electrical and Electronics Engineers (IEEE) 802.16 family of standards including IEEE 802.11 standards (WiFi), IEEE 802.16 standards (WiMax®) and others), peer-to-peer (P2P) networks, or other protocols now known or later developed.

For example, the network interface device 220 may include one or more physical jacks (e.g., Ethernet, coaxial, or phone jacks) or one or more antennas to connect to the communications network 190. In an example, the network interface device 220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the computer 200, and includes digital or analog communications signals or other intangible medium to facilitate communication of such software.

The network interface device 220 may also be a sensor interface and include any wired or wireless interface, such as a radio, for reading sensors over a wireless channel. The radio may operate using a Bluetooth®, an IEEE 802.11 standard, or any other standard for reading data from sensors over a wireless channel.

The computer 200 further comprises a map generation module 240, coupled to the link 208, that generates the various maps from the emotion indications and environmental data as described subsequently. An aggregation module 241, coupled to the link 208, aggregates the emotion indications and environmental data as described subsequently. A query module 242, coupled to the link 208, performs any query operations of other controller devices or other systems.

The computer of FIG. 2 may also include the structure for means for obtaining user emotion indications from a plurality of emotion sensors, means for obtaining respective environmental data, coincident with each user emotion indication, from a plurality of environmental sensors, means for aggregating the user emotion indications based on respective associated environmental data for each user emotion indication, and means for displaying the aggregated user emotion indications based on the respective environmental data for each user emotion indication.

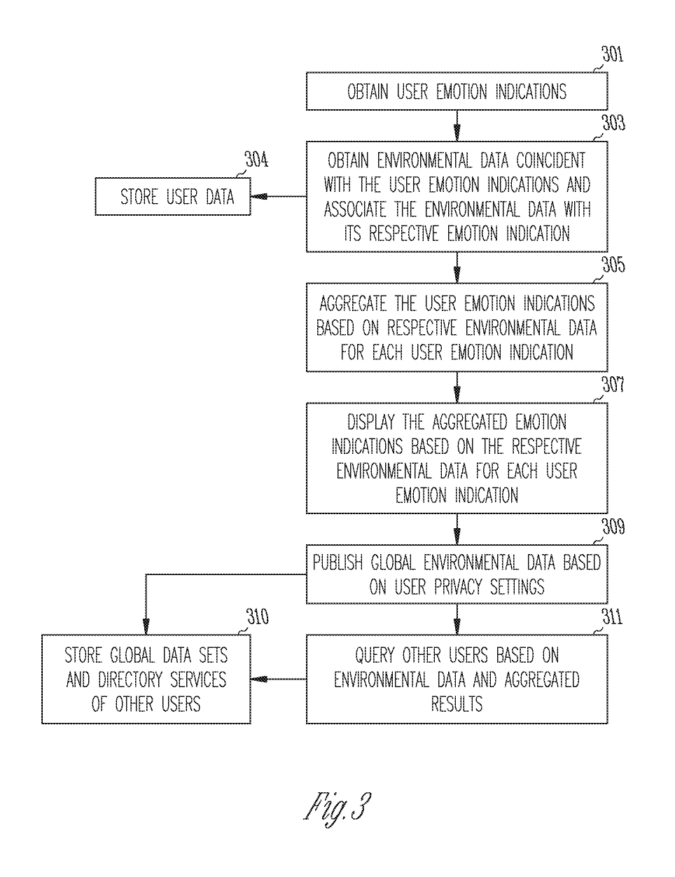

FIG. 3 illustrates a flowchart of a method for emotion mapping according to an embodiment. While elements of the following method are shown in a particular order, there is no requirement that these elements be performed in any certain order. Other than obtaining the user emotion indications and environmental data prior to storing or aggregating the data, each of the elements may be performed, or not performed, as requested by an entity.

The method obtains user emotion indications from a plurality of emotion sensors in block 301. In block 303, the environmental data coincident with those emotion indications are obtained. In an embodiment, the environmental data may be measured coincident in both time and/or space relative to the user and the user emotion that triggered the indication. In another embodiment, the user may be reacting to an event that is taking place at a different location than the user. The measured environmental data may be associated with the respective emotion indication that it was measured coincident with,

In block 304, each emotion indication and their respective environmental data may be stored. The data may be stored in the user's individual controller device or in another controller such as a computer server. In another embodiment, the emotion indications and the environmental data may be correlated at a later point.

In block 305, the user emotion indications are aggregated based on their respective environmental data for each user emotion indication. The system may use stored data from a plurality of users in aggregating the data. In block 307, the aggregated emotion indications of the plurality of users may be displayed based on the respective environmental data for each user emotion indication as well as the each user's privacy setting. The display may be in the form of a heatmap (i.e., a graphical representation of data where the individual values contained in a matrix are represented as colors), a temporal map (i.e., graphical representation of emotional data based on time), or both. The display may be a global map of the aggregated data. In an embodiment, the aggregation may be based on semantic location (e.g., preschool, library, French restaurant).

For example, using a geographical area (from GPS environmental data) as the filtering context, data may be aggregated to show the emotional status of a street, neighborhood, or city. The emotion indications and their respective environmental data may be aggregated and archived to show time variations while overlaying news, police reports, public safety reports, or seasonal change environmental data. Such data may be queried and made available to World Wide Web sites like Zillow®, that may use it to highlight the happiness of a particular neighborhood. The emotion indications may be further aggregated over a semantic context such as child versus adult or supporter of soccer team X versus supporter of soccer team Y.

Similarly, the context could be semantic locations such as schools, shopping centers, factories, businesses or other types of places. Or it could be more specific like a pediatricians' office, library or restaurant.

Another vector of querying could be by emotion or demographics or even a sequence of events. For example, an application may query the data and use it to display where, when, and why people experience fear, frustration, or sadness. These emotions could show patterns that might be of interest to the public. On a more personal and specific level, an individual could query their own data or the data of a loved one if allowed to do so based on that person's personal privacy settings. They could introspect, analyze, find correlations, or even create an alert system to help the loved one through difficult situations.

Similarly, an application may be created to show someone's effect on others. This could help a parent understand whether they are alienating their children, or a manager reach out to employees and be mindful of creating a positive environment, preserving the privacy of individuals since the aggregated data would show a pattern at the office rather than person X vs. person Y.

The system could also identify anomalies (temporal, geographical, etc.) and highlight that to users, or show change in trends over time. For example, the system may learn and detect changes in things that a user has discovered in the past and show these proactively to the user (e.g., that overall happiness in Zurich is increasing or that people immediately experience anxiety before retirement).

The aggregated data (e.g., emotion indications and their respective associated environmental data) may also be used to predict the effect of an event on an entire population or just an individual based on previous behavior. Such data may be helpful to leaders on how to help people and generate motivation or other global positive changes. For example, urban development might use the aggregated data to place parks, colorful paintings, or public transportation where an emotion indication trends toward sadness.

The aggregated data may also be used for urban planning, medical evaluation of the user or users, to suggest lifestyle changes to a user, to predict an emotional mood of a geographical area (e.g., city) based on the emotional mood of other geographical areas (e.g., other cities), or to provide an indication of emotional health of a group of users based on lifestyle changes

In block 309, global environmental data may be published based on user privacy settings. This data may be stored as global data sets or directory services of other users, based on those user's privacy settings, as seen in block 310. In block 311, one user's controller, the server/gateway controllers, or other controllers in the system may query other users based on the environmental data or aggregated results.

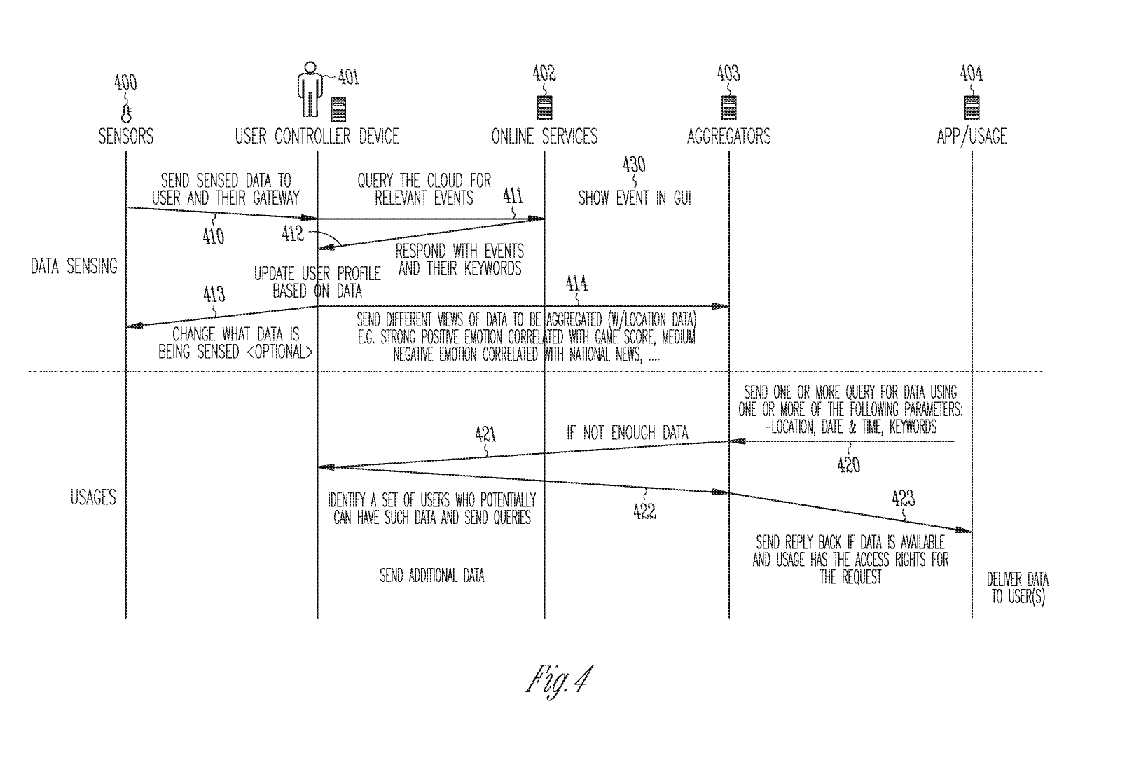

FIG. 4 illustrates a flow diagram of a method for emotion mapping, according to various embodiments. This is only one example of a usage of the method for emotion mapping.

At 410, sensed data is acquired. The sensors 400 may be read by the user's controller device 401 or gateway or their data may be sent to the user's controller device 401 or gateway. The controller device 401 thus comprises means for acquiring data from a plurality of sensors. As discussed previously, the sensors may include emotion sensors to read emotion indications of the user at a particular time, such as microphones to pick up her voice response to an event, an image sensor to pick up her facial expressions in response to the event, tactile sensors to pick up her typing of messages in response to the event, or other such emotion sensors. The sensors 400 may also include the environmental sensors surrounding the user 401 that pick up on what is happening around the user 401 in response to the event at the time.

At 411, the user's controller device 401 queries online services (e.g., news, weather) of a cloud server 402 for relevant events. The controller device 401 thus comprises means for querying the online service. The online services of the cloud server 402 may display the event in a graphical user interface format.

At 412, the cloud server 402 responds with the relevant events and the event's associated keywords. The user's controller device 401 may then update the user profile based on the received data from the cloud server 402 and the sensors 400. The user's controller device 401 thus comprises means for receiving data including emotion indications and environmental data.

At 413, the user's updated profile may change what data is being sensed by the sensors 400. This step is an optional step since the user's controller device 401 may continue to read or get data from all of the sensors 400. At 414, different views of the emotion data and associated environmental data may be transmitted to aggregators computer servers, means for aggregating) 403 for aggregating with location data. For example, strong positive emotion indications (e.g., yelling positive words) may be correlated with a game score. Medium negative emotion indications (e.g., negative facial expression) may be correlated with national news from the online services 402. The user's controller device 401 thus comprises means for updating the user profile.

At 420, an application 404 running on a computer or other electronic device may attempt to use the data from the aggregators. The user's controller device 401 may thus comprise means for querying the aggregator. The application 404 sends one or more queries for the data using one or more parameters such as location, date, time, and/or keywords. The aggregators 403 may determine that there is not enough data for the one or more queries for data and, at 421, send queries to one or more users' controller devices 401 who potentially have the needed data. The one or more users' may respond, at 422 to the aggregators 403 with the additional requested data. At 423, the aggregators 403 transmits the requested data to the application 404 for use in the application for display by the application 404. This data may only be transmitted if the data was found to be available and the application has the access rights for the request, as determined by user privacy settings.

### Additional Notes & Examples:

Example 1 is a system for mapping user emotions, the system comprising: a plurality of sensors for measuring user emotion indications; a sensor interface; and an emotion mapping module coupled to the plurality of sensors and the sensor interface, the emotion map module to read the plurality of sensors to obtain at least one user emotion indication and to obtain environmental data, from the sensor interface, coincident with the at least one user emotion indication, the emotion mapping module further associates the obtained environmental data with the at least one user emotion indication.

In Example 2, the subject matter of Example 1 optionally includes wherein the plurality of sensors comprise user wearable sensors.

In Example 3, the subject matter of any one or more of Examples 1-2 optionally include wherein the plurality of sensors comprise mobile device sensors.

In Example 4, the subject matter of any one or more of Examples 1-3 optionally include wherein the emotion mapping module obtains the environmental data from the sensor interface that is coupled to a plurality of environmental sensors that comprise air quality sensors, temperature sensors, humidity sensors, light sensors, or barometric sensors.

In Example 5, the subject matter of any one or more of Examples 1-4 optionally include wherein the plurality of sensors comprise image sensors, audio sensors, temperature sensors, heart rate sensors, tactile sensors, sound sensors.

In Example 6, the subject matter of any one or more of Examples 1-5 optionally include an aggregation module coupled to the emotion mapping module to aggregate the user emotion indications of a plurality of users based on the environmental data associated with each user emotion indication.

In Example 7, the subject matter of Example 6 optionally includes a map generation module coupled to the emotion mapping module to generate a temporal map of the user emotion indications of the plurality of users based on the environmental data associated with each user emotion indication.

In Example 8, the subject matter of Example 7 optionally includes wherein the map generation module is further to generate a heatmap of the user emotions of the plurality of users based on the environmental data associated with each user emotion indication.

In Example 9, the subject matter of any one or more of Examples 6-8 optionally include a query module coupled to the emotion mapping module to query a plurality of controller devices based on environmental data associated with each user emotion indication, wherein the plurality of controllers comprise an aggregate of user emotion indications of the plurality of users, each user emotion indication associated with respective environmental data.

In Example 10, the subject matter of Example 9 optionally includes wherein the aggregation module is further to transmit the aggregate of user emotion indications with respective associated environmental data to a second plurality of controller devices based on user privacy settings associated with each user.

In Example 11, the subject matter of any one or more of Examples 1-10 optionally include wherein the emotion mapping module is further to associate a user privacy setting with the at least one user emotion indication and associated environmental data.

Example 12 is a controller device for mapping user emotions, the controller device comprising: a sensor interface; an emotion mapping module, coupled to the sensor interface, the emotion map module to read the sensor interface to obtain at least one user emotion indication and to obtain environmental data coincident with the at least one user emotion indication, the emotion mapping module further associates the obtained environmental data with the at least one user emotion indication.

In Example 13, the subject matter of Example 12 optionally includes wherein the sensor interface is a radio that couples to a plurality of sensors over a wireless channel.

In Example 14, the subject matter of Example 13 optionally includes wherein the radio is a Bluetooth radio.

In Example 15, the subject matter of any one or more of Examples 12-14 optionally include wherein the sensor interface is a wired connection.

Example 16 is a method for mapping user emotions, the method comprising: obtaining user emotion indications from a plurality of emotion sensors; obtaining respective environmental data, coincident with each user emotion indication, from a plurality of environmental sensors; aggregating the user emotion indications for a plurality of users based on respective associated environmental data for each user emotion indication; and displaying the aggregated user emotion indications based on the respective environmental data for each user emotion indication.

In Example 17, the subject matter of Example 16 optionally includes wherein obtaining the respective environmental data coincident with each user emotion indication comprises obtaining the respective environmental data coincident in time and space with each user emotion indication.

In Example 18, the subject matter of any one or more of Examples 16-17 optionally include wherein displaying the aggregated user emotion indications comprises displaying a geographical heatmap of the aggregated user emotion indications based on the respective environmental data coincident with each user emotion indication,

In Example 19, the subject matter of any one or more of Examples 16-18 optionally include wherein displaying the aggregated user emotion indications comprises displaying the aggregated user emotion indications based on user privacy settings for each user,

In Example 20, the subject matter of any one or more of Examples 16-19 optionally include wherein aggregating the user emotion indications comprises aggregating the user emotion indications over a predetermined geographical area.

In Example 21, the subject matter of any one or more of Examples 16-20 optionally include storing the user emotion indications with respective associated environmental data on a user device.

In Example 22, the subject matter of Example 21 optionally includes determining information related to applications being used by a user on the device coincident with obtaining the user emotion indications.

In Example 23, the subject matter of Example 22 optionally includes the device preventing access to the information related to applications being used by the user in response to user privacy settings on the device.

In Example 24, the subject matter of any one or more of Examples 22-23 optionally include the device downloading the user emotion indications and respective associated environmental data to a server.

In Example 25, the subject matter of any one or more of Examples 22-24 optionally include the device transmitting the user emotion indications and respective associated environmental data to a querying application based on user privacy settings on the device.

In Example 26, the subject matter of any one or more of Examples 16-25 optionally include wherein aggregating the user emotion indications comprises aggregating the user emotion indications over a semantic context.

In Example 27, the subject matter of any one or more of Examples 16-26 optionally include wherein aggregating the user emotion indications comprises aggregating the user emotion indications for urban planning.

In Example 28, the subject matter of any one or more of Examples 16-27 optionally include wherein aggregating the user emotion indications comprises aggregating the user emotion indications for medical evaluation of the user.

In Example 29, the subject matter of any one or more of Examples 16-28 optionally include wherein aggregating the user emotion indications comprises aggregating the user emotion indications to suggest lifestyle changes to the user.

In Example 30, the subject matter of any one or more of Examples 16-29 optionally include wherein aggregating the user emotion indications comprises aggregating the user emotion indications to predict an emotional mood of a geographical area based on the emotional mood of other geographical areas.

In Example 31, the subject matter of any one or more of Examples 16-30 optionally include wherein aggregating the user emotion indications comprises aggregating the user emotion indications to provide an indication of emotional health of a group of users based on lifestyle changes.

Example 32 is at least one computer-readable medium comprising instructions for initiating emotion mapping that, when executed by a computer, cause the computer to perform any one of the method Examples 16-31.

Example 33 is an apparatus comprising means for performing any of the methods of Examples 16-31.

Example 34 is a controller device for mapping user emotions, the controller device comprising: means for obtaining user emotion indications from a plurality of emotion sensors; means for obtaining respective environmental data, coincident with each user emotion indication, from a plurality of environmental sensors; means for aggregating the user emotion indications based on respective associated environmental data for each user emotion indication; and means for displaying the aggregated user emotion indications based on the respective environmental data for each user emotion indication.

In Example 35, the subject matter of Example 34 optionally includes wherein obtaining the respective environmental data coincident with each user emotion indication comprises obtaining the respective environmental data coincident in time and space with each user emotion indication.

In Example 36, the subject matter of any one or more of Examples 34-35 optionally include wherein displaying the aggregated user emotion indications comprises displaying a geographical heatmap of the aggregated user emotion indications based on the respective environmental data coincident with each user emotion indication.

In Example 37, the subject matter of any one or more of Examples 34-36 optionally include wherein displaying the aggregated user emotion indications comprises displaying the aggregated user emotion indications based on user privacy settings for each user.

In Example 38, the subject matter of any one or more of Examples 34-37 optionally include wherein aggregating the user emotion indications comprises aggregating the user emotion indications over a predetermined geographical area.

In Example 39, the subject matter of any one or more of Examples 34-38 optionally include storing the user emotion indications with respective associated environmental data on a user device.

In Example 40, the subject matter of Example 39 optionally includes determining information related to applications being used by a user on the device coincident with obtaining the user emotion indications.

In Example 41, the subject matter of Example 40 optionally includes the device preventing access to the information related to applications being used by the user in response to user privacy settings on the device.

In Example 42, the subject matter of any one or more of Examples 40-41 optionally include the device downloading the user emotion indications and respective associated environmental data to a server.

In Example 43, the subject matter of any one or more of Examples 40-42 optionally include the device transmitting the user emotion indications and respective associated environmental data to a querying application based on user privacy settings on the device.

In Example 44, the subject matter of any one or more of Examples 34-43 optionally include wherein aggregating the user emotion indications comprises aggregating the user emotion indications over a semantic context.

In Example 45, the subject matter of any one or more of Examples 34-44 optionally include wherein aggregating the user emotion indications comprises aggregating the user emotion indications for urban planning.

In Example 46, the subject matter of any one or more of Examples 34-45 optionally include wherein aggregating the user emotion indications comprises aggregating the user emotion indications for medical evaluation of the user.

In Example 47, the subject matter of any one or more of Examples 34-46 optionally include wherein aggregating the user emotion indications comprises aggregating the user emotion indications to suggest lifestyle changes to the user.

In Example 48, the subject matter of any one or more of Examples 34-47 optionally include wherein aggregating the user emotion indications comprises aggregating the user emotion indications to predict an emotional mood of a geographical area based on the emotional mood of other geographical areas.

In Example 49, the subject matter of any one or more of Examples 34-48 optionally include wherein aggregating the user emotion indications comprises aggregating the user emotion indications to provide an indication of emotional health of a group of users based on lifestyle changes.

Example 50 is a method for emotion mapping, the method comprising: acquiring data from a plurality of sensors, the data comprising emotion indications, that occurred during a time, and associated environmental data that occurred during the time; querying an online service for data regarding events that occurred during the time; receiving the data regarding the events and associated keywords; and updating a user profile based on the data regarding the events, the emotion indications, and the associated environmental data.

In Example 51, the subject matter of Example 50 optionally includes wherein acquiring the data from the plurality of sensors comprises reading the sensors.

In Example 52, the subject matter of any one or more of Examples 50-51 optionally include wherein acquiring the data from the plurality of sensors comprises receiving the data transmitted by the sensors.

In Example 53, the subject matter of any one or more of Examples 50-52 optionally include acquiring additional data based on the updated user profile.

Example 54 is at least one computer-readable medium comprising instructions, when executed by a computer, cause the computer to perform any one of the method Examples 50-53.

Example 55 is an apparatus comprising means for performing any of the methods of Example 50-53.

Example 56 is a method for emotion mapping, the method comprising: querying an aggregator for emotion indications and associated environmental data based on at least one of location, date, time, or keywords associated with the emotion indications and environmental data; receiving the emotion indications and associated environmental data based on user privacy settings; and displaying the received emotion indications and associated environmental data.

In Example 57, the subject matter of Example 56 optionally includes querying the aggregator for emotion indications and associated environmental data based on semantic location.

In Example 58, the subject matter of any one or more of Examples 56-57 optionally include wherein displaying the received emotion indications and associated environmental data comprises displaying the received emotion indications and associated environmental data while overlaying news, police reports, public safety reports, or seasonal change environmental data.

Example 59 is at least one computer-readable medium comprising instructions, when executed by a computer, cause the computer to perform any one of the method Examples 56-58.

Example 60 is an apparatus comprising means for performing any of the methods of Example 56-58.

Example 61 is a controller device for emotion mapping, the method comprising: means for acquiring data from a plurality f sensors, the data comprising emotion indications, that occurred during a time, and associated environmental data that occurred during the time; means for querying an online service for data regarding events that occurred during the time; means for receiving the data regarding the events and associated keywords; and means for updating a user profile based on the data regarding the events, the emotion indications, and the associated environmental data.

In Example 62, the subject matter of Example 61 optionally includes wherein the means for acquiring the data from the plurality of sensors comprises means for reading the sensors.

In Example 63, the subject matter of any one or more of Examples 61-62 optionally include wherein the means for acquiring the data from the plurality of sensors comprises means for receiving the data transmitted by the sensors.

In Example 64, the subject matter of any one or more of Examples 61-63 optionally include means for acquiring additional data based on the updated user profile.

Example 65 is a controller device for emotion mapping, the method comprising: means for querying an aggregator for emotion indications and associated environmental data based on at least one of location, date, time, or keywords associated with the emotion indications and environmental data; means for receiving the emotion indications and associated environmental data based on user privacy settings; and means for displaying the received emotion indications and associated environmental data.

In Example 66, the subject matter of any one or more of Example 65 optionally includes means for querying the aggregator for emotion indications and associated environmental data based on semantic location.

In Example 67, the subject matter of any one or more of Examples 65-66 optionally include means for displaying the received emotion indications and associated environmental data while overlaying news, police reports, public safety reports, or seasonal change environmental data.

Example 68 is at least one computer-readable medium comprising instructions for mapping user emotions that, when executed by a computer, cause the computer to obtain user emotion indications from a plurality of emotion sensors; obtain respective environmental data, coincident with each user emotion indication, from a plurality of environmental sensors; aggregate the user emotion indications for a plurality of users based on respective associated environmental data for each user emotion indication; and display the aggregated user emotion indications based on the respective environmental data for each user emotion indication.

In Example 69 the subject matter of Example 68 optionally include the instructions further causing the computer to prevent access to the information related to applications being used by the user in response to user privacy settings on the computer.

In Example 70 the subject matter of any one or more of Examples 68-69 optionally include the instructions further cause the computer to download the user emotion indications and respective associated environmental data to a server.

In Example 71 the subject matter of any one or more of Examples 68-70 optionally include the instructions further cause the computer to obtain the emotion indications from the plurality of sensors by receiving the data transmitted by the sensors.

Example 72 is at least one computer-readable medium comprising instructions for mapping user emotions that, when executed by a computer, cause the computer to: obtain user emotion indications from a plurality of emotion sensors; obtain respective environmental data, coincident with each user emotion indication, from a plurality of environmental sensors; aggregate the user emotion indications for a plurality of users based on respective associated environmental data for each user emotion indication; and display the aggregated user emotion indications based on the respective environmental data for each user emotion indication.

In Example 73, the subject matter of Example 72 optionally includes wherein the instructions further cause the computer to obtain the respective environmental data coincident in time and space with each user emotion indication,

In Example 74, the subject matter of any one or more of Examples 72-73 optionally include wherein the instructions further cause the computer to display the aggregated user emotion indications by displaying a geographical heatmap of the aggregated user emotion indications based on the respective environmental data coincident with each user emotion indication.

In Example 75, the subject matter of any one or more of Examples 72-74 optionally include wherein the instructions further cause the computer to display the aggregated user emotion indications by displaying the aggregated user emotion indications based on user privacy settings for each user.

In Example 76, the subject matter of any one or more of Examples 72-75 optionally include wherein the instructions further cause the computer to aggregate the user emotion indications by aggregating the user emotion indications over a predetermined geographical area.

In Example 77, the subject matter of any one or more of Examples 72-76 optionally include wherein the instructions further cause the computer to store the user emotion indications with respective associated environmental data on a user device.

In Example 78, the subject matter of any one or more of Examples 72-77 optionally include wherein the instructions further cause the computer to determine information related to applications being used by a user on the device coincident with obtaining the user emotion indications.

In Example 79, the subject matter of any one or more of Examples 72-78 optionally include wherein the instructions further cause the computer to prevent access to the information related to applications being used by the user in response to user privacy settings on the device.

In Example 80, the subject matter of any one or more of Examples 72-79 optionally include wherein the instructions further cause the computer to download the user emotion indications and respective associated environmental data to a server.

In Example 81, the subject matter of any one or more of Examples 72-80 optionally include wherein the instructions further cause the computer to download the user emotion indications and respective associated environmental data to a querying application based on user privacy settings on the device.

In Example 82, the subject matter of any one or more of Examples 72-81 optionally include wherein the instructions further cause the computer to aggregate the user emotion indications by aggregating the user emotion indications over a semantic context.

In Example 83, the subject matter of any one or more of Examples 72-82 optionally include wherein the instructions further cause the computer to aggregate the user emotion indications by aggregating the user emotion indications for urban planning.

In Example 84, the subject matter of any one or more of Examples 72-83 optionally include wherein the instructions further cause the computer to aggregate the user emotion indications by aggregating the user emotion indications for medical evaluation of the user.

In Example 85, the subject matter of any one or more of Examples 72-84 optionally include wherein the instructions further cause the computer to aggregate the user emotion indications by aggregating the user emotion indications to suggest lifestyle changes to the user.

In Example 86, the subject matter of any one or more of Examples 72-85 optionally include wherein the instructions further cause the computer to aggregate the user emotion indications by aggregating the user emotion indications to predict an emotional mood of a geographical area based on the emotional mood of other geographical areas.

In Example 87, the subject matter of any one or more of Examples 72-86 optionally include wherein the instructions further cause the computer to aggregate the user emotion indications by aggregating the user emotion indications to provide an indication of emotional health of a group of users based on lifestyle changes.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

What is claimed is:

1. A controller device for mapping user emotions, the controller device comprising:
a sensor interface to receive sensor data comprising user emotion indications and environmental data from a plurality of sensors; and
an emotion mapping module, coupled to the sensor interface, the emotion map module to read the sensor interface to obtain at least one user emotion indication and to obtain environmental data coincident with the at least one user emotion indication, the emotion mapping module further associates the obtained environmental data with the at least one user emotion indication and displays the association.

2. The system of claim 1, wherein the plurality of sensors comprise user wearable sensors.

3. The system of claim 1, wherein the plurality of sensors comprise mobile device sensors.

4. The system of claim 1, wherein the emotion mapping module obtains the environmental data from the sensor interface that is coupled to a plurality of environmental sensors that comprise air quality sensors, temperature sensors, humidity sensors, light sensors, or barometric sensors.

5. The system of claim 1, wherein the plurality of sensors comprise image sensors, audio sensors, temperature sensors, heart rate sensors, tactile sensors, sound sensors.

6. The system of claim 1, further comprising an aggregation module coupled to the emotion mapping module to aggregate the user emotion indications of a plurality of users based on the environmental data associated with each user emotion indication.

7. The system of claim 6, further comprising a map generation module coupled to the emotion mapping module to generate a temporal map of the user emotion indications of the plurality of users based on the environmental data associated with each user emotion indication.

8. The system of claim 7, wherein the map generation module is further to generate a heatmap of the user emotions of the plurality of users based on the environmental data associated with each user emotion indication.

9. The system of claim 6, further comprising a query module coupled to the emotion mapping module to query a plurality of controller devices based on environmental data associated with each user emotion indication, wherein the plurality of controllers comprise an aggregate of user emotion indications of the plurality of users, each user emotion indication associated with respective environmental data.

10. The system of claim 9, wherein the aggregation module is further to transmit the aggregate of user emotion indications with respective associated environmental data to a second plurality of controller devices based on user privacy settings associated with each user.

11. The system of claim wherein the emotion mapping module is further to associate a user privacy setting with the at least one user emotion indication and associated environmental data.

12. A computer-implemented r emotion mapping, the method comprising:
acquiring data from a plurality of sensors, the data comprising emotion indications, that occurred during a time, and associated environmental data that occurred during the time;
querying an online service for data regarding events that occurred during the time;
receiving the data regarding the events and associated keywords; and
updating a user profile based on the data regarding the events, the emotion indications, and the associated environmental data.

13. The method of claim 12, wherein acquiring the data from the plurality of sensors comprises reading the sensors.

14. The method of claim 12, wherein acquiring the data from the plurality of sensors comprises receiving the data transmitted by the sensors.

15. The method of claim 12, further comprising acquiring additional data based on the updated user profile.

16. A computer-implemented method for mapping user emotions, the method comprising:
obtaining user emotion indications from a plurality of emotion sensors;
obtaining respective environmental data, coincident with each user emotion indication, from a plurality of environmental sensors;
aggregating the user emotion indications for a plurality of users based on respective associated environmental data for each user emotion indication; and
displaying the aggregated user emotion indications based on the respective environmental data for each user emotion indication.

17. The method of claim 16, wherein obtaining the respective environmental data coincident with each user emotion indication comprises obtaining the respective environmental data coincident in time and space with each user emotion indication.

18. The method of claim 16, wherein displaying the aggregated user emotion indications comprises displaying a geographical heatmap of the aggregated user emotion indications based on the respective environmental data coincident with each user emotion indication.

19. The method of claim 18, wherein displaying the aggregated user emotion indications comprises displaying the aggregated user emotion indications based on user privacy settings for each user.

20. The method of claim 16, wherein aggregating the user emotion indications comprises aggregating the user emotion indications over a predetermined geographical area.

21. The method of claim 16, further comprising storing the user emotion indications with respective associated environmental data on a user device.

22. At least one computer-readable medium comprising instructions for mapping user emotions that, when executed by a computer, cause the computer to:
obtain user emotion indications from a plurality of emotion sensors;
obtain respective environmental data, coincident with each user emotion indication, from a plurality of environmental sensors;
aggregate the user emotion indications for a plurality of users based on respective associated environmental data for each user emotion indication; and
display the aggregated user emotion indications based on the respective environmental data for each user emotion indication.

23. The computer-readable medium of claim 22, wherein the instructions further cause the computer to prevent access to the information related to applications being used by the user in response to user privacy settings on the computer.

24. The computer-readable medium of claim 22, wherein the instructions further cause the computer to download the user emotion indications and respective associated environmental data to a server.

25. The computer-readable medium of claim 222 wherein the instructions further cause the computer to obtain the emotion indications from the plurality of sensors by receiving the data transmitted by the sensors.
